(19) United States
(12) Patent Application Publication
(10) Pub. No.: US 2020/0093887 A1
(43) Pub. Date: Mar. 26, 2020

(54) OPHTHALMIC TREATMENT COMPOSITION AND VEHICLE FOR DELIVERY OF PHARMACEUTICAL SUBSTANCES OR THERAPEUTIC AGENTS

(71) Applicant: Paul Gavaris, Bethesda, MD (US)

(72) Inventor: Paul Gavaris, Bethesda, MD (US)

(21) Appl. No.: 16/566,624

(22) Filed: Sep. 10, 2019

Related U.S. Application Data
(63) Continuation-in-part of application No. 16/385,449, filed on Apr. 16, 2019, now Pat. No. 10,406,203.
Application No. 16/385,449 is a division of application No. 15/292,718, filed on Oct. 13, 2016, now Pat. No. 10,286,035.
(60) Provisional application No. 62/241,462, filed on Oct. 14, 2015.

(51) Int. Cl. A61K 38/13; A61P 27/02; A61K 9/08; A61K 31/01; A61K 9/00; A61K 31/496; A61K 31/573; A61K 31/7048; A61K 47/06

(52) U.S. Cl. CPC A61K 38/13; A61P 27/02; A61K 9/08; A61K 31/01; A61K 47/06; A61K 31/496; A61K 31/573; A61K 31/7048; A61K 9/0048

(57) ABSTRACT

An ophthalmic treatment composition for the treatment of dry eye consists essentially of squalane in an amount by weight of 1% to 100%; and mineral oil in an amount by weight of 0% to 99%. Preferably, the mineral oil is a light mineral oil or mixture of light mineral oils and is present in an amount by weight of 1% to 10%. The composition is effectively devoid of water, preservatives, emulsifiers and dispersing agents. A related ophthalmic treatment composition comprises squalane in an amount by weight of 1% to 99% and mineral oil in an amount by weight of 0% to 99% and at least one hydrophobic or lipophilic pharmaceutical substance in an amount by weight of 0.01% to 5%, effective for treatment of at least one ophthalmic condition.

### CROSS-REFERENCE TO RELATED APPLICATION

This application is a continuation-in-part of application Ser. No. 16/385,449, now U.S. Pat. No. 10,406,203, filed Apr. 16, 2019 as division of application Ser. No. 15/292,718 filed Oct. 13, 2016, now U.S. Pat. No. 10,286,035. This application also claims the benefit of U.S. Provisional Patent Application No. 62/241,462 filed Oct. 14, 2015.

### BACKGROUND OF THE INVENTION

This invention relates to a composition and associated method for treatment of certain ophthalmic conditions. The invention also relates to a composition and associated method for treatment of a number of disease states via an ophthalmic delivery process. The invention further relates to a composition for use as a delivery vehicle or carrier in the manufacture of a composition containing a pharmaceutical substance or therapeutic agent.

Dry eye syndrome is characterized by sensations of dryness, burning, and a sandiness or grittiness that can worsen during the day. Symptoms are typically described as burning, itchy, scratchy, stingy or tired eyes. Other symptoms include pain, redness, a pulling sensation, and pressure behind the eye. The damage to the eye surface resulting from dry eye increases discomfort and sensitivity to bright light and both eyes usually are affected, but symptoms may be occur asymmetrically.

Having dry eyes for a prolonged period of time can lead to tiny abrasions on the surface of the eyes. In advanced cases, the epithelium undergoes pathologic changes, namely squamous metaplasia and loss of goblet cells sometimes due to activation of T cells directed. Some severe cases result in thickening of the corneal surface, conical erosion, punctate keratopathy, epithelial defects, corneal ulceration, corneal neovascularization, corneal scarring, corneal thinning, and even corneal perforation. An abnormality of any one of the three layers of tears which produces an unstable tear film, may result in symptoms of keratitis sicca.

Another significant benefit of this invention is to provide a preservative-free vehicle for ophthalmic medications. With such chronic conditions as glaucoma, allergies, uveitis, and dry eye and with other frequent needs, the use of ophthalmic medications containing preservatives is counter-productive as they often produce serious side effects to the eye and adnexal structures and accordingly should to be stopped. The preservatives being surfactants damage the epithelium with each use in an infected or already allergic conjunctiva thus reducing the full effectiveness of the desired therapy. Therefore this invention affords significant benefit on 2 major venues, namely, it eliminates the deleterious effects of preservatives, and by its inherent therapeutic antioxidant and emollient properties supplements the therapeutic goal. Another benefit of the present invention is that the medications can be delivered in a single daily dose at night, easily in a dropper bottle form that patients find convenient and economical in lieu of the single-use plastic dropperettes that can scratch the corneas.

There is a need for an improved method and composition for the treatment of chronic ophthalmic conditions such as dry eye (DED) and glaucoma. Conventional treatments require frequent use of eye drops instilled into the eye. There is a high level of interest in using food oils, such as olive oil, in ocular formulas for dry eye treatment or other chronic ophthalmic diseases. Compositions have been proposed which include castor oil to facilitate spreading of the composition over the surface of the eye. See, e.g., U.S. Patent Application Publication No. 2008/0070834 and U.S. Pat. No. 8,679,554.

The great majority of the compositions used include either a preservative such as polysorbate 80 (U.S. Pat. No. 8,679,554), benzalkonium chloride or thimerosal and/or an emulsifier or surfactant (Publication No. 2008/0070834). The preservatives and emulsifiers are either toxic to the epithelial cells or are allergenic to the patients. Over time this mild toxicity when induced 2-3 times a day can create serious redness, tissue changes such as conjunctival contracture or subconjunctival fibrosis, and actual damage to already compromised tissue. Thus any method of drug delivery which does not contain these preservatives would be much preferred and would be ready substitutes for present vehicles for eye drug delivery or have inherent emollient benefits as well.

### OBJECTS OF THE INVENTION

It is an object of the present invention to provide an improved composition for the treatment of dry eye, the composition serving either as a medication delivery vehicle and/or as an emollient.

A more specific object of the present invention is to provide a composition for the treatment of dry eye which does not include even incidental amounts of preservatives and emulsifying agents.

A related object of the present invention is to provide an improved vehicle for delivering a therapeutic agent primarily to the corneal epithelium and adnexal structures.

An associated specific object of the invention is to provide a medicament delivery vehicle or composition which does not include even incidental amounts of preservatives and emulsifying agents.

These and other objects of the invention will be apparent from the descriptions herein. Although every object of the invention is attained in at least one embodiment of the invention, there is not necessarily any embodiment which attains all of the objects of the invention.

### SUMMARY OF THE INVENTION

The present invention is directed to a composition for use in the treatment of undesirable ophthalmic conditions including dry eye and various disease states such as allergic conjunctivitis, atopic dermatitis, or eczematous change, or as a protective from excess tears. The composition comprises 100% squalane alone or optionally in combination with mineral oil, preferably a light mineral oil (N.F.). It is contemplated that the composition is essentially devoid of preservatives, emulsifiers and surfactants. Impurities may be incidentally present in the composition but only in trace amounts that have no practical, cognizable or determinable effect on the properties of the composition or its effect on human users. The composition is typically used via topical application to the eye of a mammal, suffering from dry eye, to relieve the symptoms thereof via its several properties such as its emollient properties or its inherent pharmaco-therapeutic actions.

Furthermore, the present invention contemplates a method for treating keratoconjunctivitis sicca (KCS) or dry eye comprising providing the above composition, and administering said composition topically to the ocular surface or immediate vicinity of an eye of a patient. This ophthalmic treatment composition is hypo-allergenic and of virtually zero toxicity to ophthalmic tissues.

Moreover, such a dry-eye treatment composition may be used as a delivery vehicle or carrier composition for a wide array of ophthalmic medicaments and pharmaceuticals, including but not limited to cyclosporin, prostaglandins, hydrocortisone, prednisone, prednisolone, dexamethasone, hydrophobic antibiotics, hydrophobic anti-inflammatories, hydrophobic antihistamines, hydrophobic NSAIDs, hydrophobic hormones such as estrogen, progesterone, and testosterone. Accordingly, the dry-eye treatment composition is useful as a delivery vehicle or carrier component in the manufacture of a medicament to be applied to the eye directly or indirectly via the eyelid, where the medicament includes a pharmaceutical substance or therapeutic agent in addition to the delivery vehicle or carrier. The light mineral oil is provided in an amount which enables effective solubility of the pharmaceutical component whether one or more of the exemplary therapeutic agents identified herein or any other suitable agent, medicament, or pharmaceutical substance.

Squalane is a hydrocarbon and triterpene derived by hydrogenation of squalene. Due to the complete saturation of squalane, it is not subject to auto-oxidation. The name selected for squalane by the International Union of Pure and Applied Chemistry (IUPAC) is 2,6,10,15,19,23-Hexamethyltetracosane. Squalane may be obtained from shark liver or olive oil, the latter route being preferred.

Ophthalmic treatment compositions according to the present invention have a soothing and healing effect on the eye. In the case of dry eye, the composition reduces or eliminates characteristic symptoms of dryness, burning, sandiness or grittiness, itchiness, stinging, tiredness, pain, redness, pulling sensations, pressure sensations and sensitivity to bright light or pain.

A dry-eye treatment composition or a drug delivery vehicle composition in accordance with the present invention is clear, without odor, non-allergenic or at least minimally allergenic to extremely sensitive patients. It has a soothing or comforting effect on even undiagnosed ocular conditions. The present composition is hydrophobic and therefore able to dissolve medicinal molecules such as cyclosporin and other oil soluble medicaments. The composition has a hygroscopic chemistry that naturally inhibits bacterial growth, similar to olive oil or 100% glycerin, both of which have been left open or in a container without needing a preservative to maintain sterility. The compositions according to the present invention are therefore storage stable at room temperatures.

A composition in accordance with the present invention has very high antioxidant, therapeutic and emollient properties. It may include light mineral oil, N.F., in any percentage as a diluent and solubility enhancing component to any degree, i.e. from 1% vol. per vol. to 99% vol./vol. and still retain its basic self preserving properties. The light mineral oil, N.F. (national formula), is included in a delivery vehicle or carrier composition as needed in order to provide for a desired solubility (and hence concentration) of any pharmaceutical agent or medicinal compound that have increased solubility in light mineral oil as opposed to squalane alone.

The composition can be ophthalmically (drops) applied 2-3 times a day or just once at night to deliver sufficient concentrations of a payload pharmaceutical, medicinal herb or other medicament, enabling once daily or possible less frequent dosing and still yield desired treatment of the ophthalmic condition.

The composition can be applied directly to the eyelid margin/lashes and still be medically therapeutic (overnight). A suitable applicator may be a roller, with an applicator ball or cylinder up to several millimeters in width. Other acceptable applicators are eye droppers which can naturally be used to apply the composition directly to the ocular surface, as well as to an eyelid. An alternate method of application is to apply an aliquot of the composition to a finger, e.g., a drop from an eye dropper, or a half-inch swipe of a roller (depending on the size of the roll surface) and then use the finger to transfer the composition to the eyelid which with proximate natural blinking permits migration of the oil to the eyeball. Aliquots of the composition may be provided in single-use disposable containers, such as bottles or tubes, each containing enough for a single application (if the medication required it) but not for sterilization purposes.

The composition vehicle can also be used as eye lid skin moisturizer and not be deleterious to the eye itself.

This composition serves as a delivery vehicle for oil-soluble drugs or other oils that are themselves hygroscopic sufficient to be self preserving thus not alter the basic advantages of the composition.

The present invention contemplates further compositions and related treatment methods including a method for treating ocular or peri-ocular tissue in a mammal in need thereof. This method consists of administering to an eye or surrounding tissues of the mammal in need thereof therapeutically effective amounts of a composition consisting essentially of squalane in combination with an amount of a treatment substance taken from the group consisting of 0.03% to 0.05% Tacrolimus by weight for pets and humans with atopic dermatitis and other adnexal tissue, 0.3% to 0.5% Loteprednol suspensions by weight for ocular inflammation and eye lid and facial skin disorders, 1% to 2% Hydrocortisone by weight for ocular and adnexal and facial allergic and inflammatory skin conditions, 0.5% to 1.0% Dexamethasone by weight for ocular and adnexal and facial allergic and inflammatory skin conditions, titanium dioxide powder of nanoscale particle size as eye lid sunscreen from UVA rays, zinc oxide as UVB sunscreen, about 3% tobramycin by weight as antibiotic for ocular and peri-ocular skin infections, about 3% bacitracin by weight as antibiotic to eye and peri-ocular tissue, and 3% Cefalexin by weight for ocular and per-ocular antibacterial use. These percentages are percentages by weight of the entire composition. The squalane of the composition inhibits dry eye, the composition including squalane in an amount of at least 80% volume/volume. The composition is effectively devoid of water, preservatives, emulsifiers and dispersing agents.

The composition may include mineral oil or mixture of mineral oils in an amount of 1% to 10% volume/volume.

A method in accordance with the present invention is directed to treating an ophthalmic or per-ocular skin condition in a mammal in need thereof and, wherein the ophthalmic or per-ocular skin condition is taken from the group consisting of atopic dermatitis and other adnexal tissue condition, ocular inflammation and eye lid and facial skin disorders, ocular and adnexal and facial allergic and inflammatory skin conditions, ocular and adnexal and facial allergic and inflammatory skin conditions, exposure to UVA or UVB radiation, ocular and peri-ocular skin infections. The method consists of administering to an eye or surrounding tissues of the mammal in need thereof therapeutically effective amounts of a composition consisting essentially of:

squalane in an amount of at least 80% (preferably at least 95% and up to 99%) volume/volume of the composition, wherein the squalane inhibits dry eye;

a chemical treatment substance in an amount by weight of 0.01% to 5% of the weight of the entire composition,

the composition being effectively devoid of water, preservatives, emulsifiers and dispersing agents.

The chemical treatment substance is preferably taken from the group consisting of Tacrolimus, Loteprednol, hydrocortisone Dexamethasone, titanium dioxide powder, zinc oxide, tobramycin, bacitracin and Cefalexin.

A method for treating ocular or peri-ocular tissue in a mammal in need thereof consisting of administering to the mammal in need thereof effective amounts of a composition consisting of squalane, wherein the composition including squalane in an amount of at least 80% (and preferably 95%) volume/volume, optionally including a mineral oil or mixture of mineral oils and also including a treatment agent or substance, the composition being effectively devoid of water, preservatives, emulsifiers and dispersing agents. The administering of the composition comprises topically applying an effective amount thereof to an eye or per-ocular tissue of the mammal.

### DETAILED DESCRIPTION

The following definitions are used to describe the present invention. In instances where a term is not specifically defined, the definition to be used is that which one of ordinary skill in the art would use to define that term within the context of that term's use.

The term “patient” or “subject” is used to describe an animal, especially a human patient in need, who receives medical attention, care, or treatment of the present invention.

The term “effective” is used to describe an amount of an ophthalmic composition, or a component, extract, material or solvent of a composition which is used to produce an intended effect in amount consistent with the effect desired and may vary with the effect desired or which occurs.

The term “mineral oil” or “light mineral oil” is used throughout the specification to describe any of various lubricious, hydrophobic and combustible substances obtained from mineral matter. Mineral oils for use in the present invention may include petroleum-based oil derivatives such as purified petrolatum and mineral oil, particularly light-weight oil, N.F. (national formula). Petroleum-derived oils include aliphatic or wax-based oils, aromatic or asphalt-based oils and mixed base oils. Preferred mineral oils for use in the present invention include petrolatum, mineral oil or mixtures of petrolatum and mineral oil where the amount of petrolatum to mineral oil (on a weight/weight basis) ranges from about 1:20 to about 10:1, preferably about 1:5 to about 5:1, more preferably about 1:3 to about 1:1, depending upon the end use of the emulsion composition.

The compositions of the present invention are preferably hydrophobic and lipophilic. Both squalane and mineral oils as used in the present invention are non-polar oils which are essentially hydrophobic and lipophilic.

Preferably, the pharmaceutical compositions of this invention are administered topically, that is, by direct contact with a patient's eye (via a drop) or a patient's skin surface around an eye, for instance, on an eyelid. The compositions may be applied by roller, eye dropper, finger, or pad (single use applications). Alternatively, the compositions may be applied directly to the surface of the eye via an eyedropper or possibly via a spray or aerosol.

It is contemplated that compositions of the present invention are effectively devoid of water, preservatives, emulsifiers and dispersing agents. The benefit is substantial in reducing undesirable side effects that these agents have. Drug-delivery compositions of the present invention include significant amounts only of squalane, mineral oil and a pharmaceutical compound or medicinal agent that constitutes a therapeutic payload. Any other ingredients are incidental and have no discernable or practical effect on the properties of the composition or its effectiveness in therapeutic application.

Methods for preparing dosage forms are known or will be apparent to those skilled in the art; for example, see “Remington's Pharmaceutical Sciences” (17th Ed., Mack Pub. Co, 1985). The person of ordinary skill will take advantage of favorable pharmacokinetic parameters of the pro-drug forms of the present invention, where applicable, in delivering the present compounds to a patient suffering from an ophthalmic infection, inflammation, or allergy, to maximize the intended effect of the compound.

In addition to the carrier or delivery vehicle as described herein, a composition for treating one or more disease eye conditions may contain multiple active ingredients or therapeutic agents in the treatment of any one or more of the disease states or conditions. Effective amounts or concentrations of each of the active compounds are to be included within pharmaceutical or therapeutic compositions according to the present invention. Individual therapeutic agents may be administered either sequentially or simultaneously in separate or combined pharmaceutical formulations.

When one or more of the compounds according to the present invention is used in combination with a second therapeutic agent active the dose of each compound may be either the same as or differ from that when the compound is used alone. Appropriate doses will be readily appreciated by those skilled in the art.

In method aspects according to the present invention, one or more compositions according to the present invention may be administered to a patient in the treatment or prevention of any disease state or condition previously mentioned. An effective amount of an therapeutic agent as otherwise described herein is administered to a patient exhibiting symptoms of a disease state or condition as otherwise described herein in order to treat the symptoms of the disease state and/or condition and reduce or eliminate the likelihood that the disease state or condition will escalate or worsen.

Therapeutic ophthalmic compositions according to the present invention comprise an effective amount of one or more of therapeutic agents in liquid, semi-liquid or solid form, otherwise described herein, in combination with the delivery vehicle or petrolatum ointment carrier comprising squalane and optionally mineral oil or mixture of mineral oils. At least one additional agent useful in treating an ophthalmic or other disease state or condition may be included in the composition. In this aspect of the invention, multiple compounds may be advantageously formulated to be co-administered for the prophylactic and/or therapeutic treatment of any one or more of the disease states or conditions described hereinabove.

The individual components of such combinations as described above may be administered either sequentially or simultaneously in separate or combined pharmaceutical formulations. When one or more compositions according to the present invention are used in combination with a second therapeutic agent, the dose of each may be either the same as or differ from that when the compound is used alone. Appropriate doses will be readily appreciated by those skilled in the art.

### Examples

An ophthalmic treatment composition for the treatment of dry eye consists essentially of squalane in an amount by weight of 1% to 99%; and mineral oil in an amount by weight of 1% to 99%. Preferably, the mineral oil is a light mineral oil or mixture of light mineral oils and is present in an amount by weight of 1% to 10%. The composition is effectively devoid of water, preservatives, emulsifiers and dispersing agents. Thus, any trace amounts of such substances are negligible in terms of effects on properties of the composition and effects on mammalian subjects.

A related method for treating keratoconjunctivitis sicca in a mammal in need thereof consists of administering to the mammal in need thereof therapeutically effective amounts of a composition comprising 100% squalane or optionally squalane in combination with a mineral oil or mixture of mineral oils. The composition comprises squalane in an amount by weight of 1% to 100%, and mineral oil in an amount by weight of 0% to 99%. The method of administering of the composition preferably comprises topically applying a therapeutically effective amount thereof to an eye or eyelid lashes of the mammal. The composition may be applied one to three or four times daily. The application may be effect either on mammalian subjects to which the composition is applied or on the properties of the composition including its storage stability, anti-contaminant attributes that preclude need for preservatives.

A method for treating an ophthalmic disease state or condition in a mammal in need thereof consists of administering to the mammal in need thereof therapeutically effective amounts of a composition comprising squalane in combination with a hydrophobic pharmaceutical substance in an amount by weight of 0.01% to 5% and optionally mineral oil or a mixture of mineral oils in an amount sufficient to solubilize the hydrophobic pharmaceutical substance.

As indicated above, the pharmaceutical substance may be cyclosporin, hydrocortisone, prednisone, or prednisolone in an amount by weight of 0.5%-2%, a hydrophobic antibiotic, a hydrophobic anti-inflammatory, an hydrophobic antihistamine, a hydrophobic NSAID, or a hydrophobic hormone or any mixture of these substances.

In the examples below, the mineral oil is typically light mineral oil, N.F.

### Dry Eye Treatment Compositions and Methods

### Symptoms: dry eye

Composition: 90% squalane, 10% mineral oil
Administration: 3-5 drops daily, preferably all together in the evening before bedtime

### Symptoms: dry eye

Composition: 95% squalane, 5% mineral oil
Administration: 3-5 drops daily, preferably all together in the evening before bedtime

### Symptoms: dry eye

Composition: 80% squalane, 20% mineral oil
Administration: 3-5 drops daily, preferably all together in the evening before bedtime

### Symptoms: dry eye

Composition: 90% squalane, 10% mineral oil
Administration: 1-3 drops, 3 times daily

### Symptoms: dry eye

Composition: 95% squalane, 5% mineral oil
Administration: 1-3 drops, 3 times daily

### Symptoms: dry eye

Composition: 80% squalane, 20% mineral oil
Administration: 1-3 drops, 3 times daily

### Symptoms: dry eye

Composition: 60% squalane, 40% mineral oil
Administration: 1-3 drops, 3 times daily

### Symptoms: dry eye

Composition: 20% squalane, 80% mineral oil
Administration: 3-5 drops daily, preferably all together in the evening before bedtime

### Symptoms: dry eye

Composition: 5% squalane, 95% mineral oil
Administration: 3-5 drops daily, preferably all together in the evening before bedtime

### Symptoms: dry eye

Composition: 55% squalane, 45% mineral oil
Administration: 3-5 drops daily, preferably all together in the evening before bedtime

### Symptoms: dry eye

Composition: 10% squalane, 80% mineral oil
Administration: 3-5 drops daily, preferably all together in the evening before bedtime

### Symptoms: dry eye

Composition: 40% squalane, 20% mineral oil
Administration: 3-5 drops daily, preferably all together in the evening before bedtime

### Symptoms: dry eye

Composition: 55% squalane
Administration: 3-5 drops daily, preferably all together in the evening before bedtime

### Symptoms: dry eye

Composition: 55% squalane, 25% mineral oil
Administration: 3-5 drops daily, preferably all together in the evening before bedtime

### Symptoms: dry eye

Composition: 5% squalane, 95% mineral oil
Administration: 1-3 drops, 3 times daily

### Symptoms: dry eye

Composition: 55% squalane, 45% mineral oil
Administration: 1-3 drops, 3 times daily

### Symptoms: dry eye

Composition: 10% squalane, 90% mineral oil
Administration: 1-3 drops, 3 times daily

### Symptoms: dry eye

Composition: 40% squalane, 60% mineral oil
Administration: 1-3 drops, 3 times daily

### Symptoms: dry eye

Composition: 90% squalane, 9% mineral oil, 1% cyclosporin
Administration: 3-5 drops daily, preferably all together in the evening before bedtime

### Symptoms: dry eye

Composition: 94% squalane, 5% mineral oil, 1% cyclosporin
Administration: 3-5 drops daily, preferably all together in the evening before bedtime

### Symptoms: dry eye

Composition: 93% squalane, 5% mineral oil, 2% cyclosporin
Administration: 3-5 drops daily, preferably all together in the evening before bedtime

### Symptoms: dry eye

Composition: 80% squalane, 18% mineral oil, 2% cyclosporin
Administration: 3-5 drops daily, preferably all together in the evening before bedtime

### Symptoms: dry eye

Composition: 78% squalane, 18% mineral oil, 4% cyclosporin
Administration: 3-5 drops daily, preferably all together in the evening before bedtime

### Symptoms: dry eye

Composition: 70% squalane, 27% mineral oil, 3% cyclosporin
Administration: 3-5 drops daily, preferably all together in the evening before bedtime

### Symptoms: dry eye

Composition: 88% squalane, 10% mineral oil, 2% cyclosporin
Administration: 1-3 drops, 3 times daily

### Symptoms: dry eye

Composition: 92% squalane, 5% mineral oil, 3% cyclosporin
Administration: 1-3 drops, 3 times daily

### Symptoms: dry eye

Composition: 80% squalane, 16% mineral oil, 4% cyclosporin
Administration: 1-3 drops, 3 times daily

### Symptoms: dry eye

Composition: 80% squalane, 19% mineral oil, 1% cyclosporin
Administration: 1-3 drops, 3 times daily

### Symptoms: dry eye

Composition: 50% squalane, 49% mineral oil, 1% cyclosporin
Administration: 3-5 drops daily, preferably all together in the evening before bedtime

### Symptoms: dry eye

Composition: 60% squalane, 38% mineral oil, 2% cyclosporin
Administration: 3-5 drops daily, preferably all together in the evening before bedtime

### Symptoms: dry eye

Composition: 75% squalane, 23% mineral oil, 2% cyclosporin
Administration: 3-5 drops daily, preferably all together in the evening before bedtime

### Symptoms: dry eye

Composition: 18% squalane, 80% mineral oil, 2% cyclosporin
Administration: 3-5 drops daily, preferably all together in the evening before bedtime

### Symptoms: dry eye

Composition: 40% squalane, 59% mineral oil, 1% cyclosporin
Administration: 3-5 drops daily, preferably all together in the evening before bedtime

### Symptoms: dry eye

Composition: 55% squalane, 42% mineral oil, 3% cyclosporin
Administration: 3-5 drops daily, preferably all together in the evening before bedtime

### Symptoms: dry eye

Composition: 50% squalane, 49% mineral oil, 1% cyclosporin
Administration: 1-3 drops, 3 times daily

### Symptoms: dry eye

Composition: 50% squalane, 46% mineral oil, 4% cyclosporin
Administration: 1-3 drops, 3 times daily

### Symptoms: dry eye

Composition: 55% squalane, 43% mineral oil, 2% cyclosporin
Administration: 1-3 drops, 3 times daily

### Symptoms: dry eye

Composition: 10% squalane, 88% mineral oil, 2% cyclosporin
Administration: 1-3 drops, 3 times daily

### Symptoms: dry eye

Composition: 40% squalane, 59% mineral oil, 1% cyclosporin
Administration: 1-3 drops, 3 times daily

### Symptoms: dry eye

Composition: 55% squalane, 44% mineral oil, 1% cyclosporin
Administration: 1-3 drops, 3 times daily

### Symptoms: dry eye

Composition: 55% squalane, 43% mineral oil, 2% cyclosporin
Administration: 1-3 drops, 3 times daily

### Hydrocortisone

Hydrocortisone or hydrocortisone acetate belongs to a group of medicines called corticosteroids and is used to relieve redness and swelling, to treat non-infected conditions such as swelling of parts of the eye and injury to the cornea (or conjunctiva). In addition, hydrocortisone is used to treat viral infection (by herpes zoster) of the eye. Dosage schedule as exemplarily 3-5 drops: or once daily at bedtime, until symptoms dissipate.

TABLE 1
Hydrocortisone

| Example No. | Squalane | Mineral Oil | Hydrocortisone |
|---|---|---|---|
| 1 | 98% | 1.99% | 0.01% |
| 2 | 90% | 9.99% | 0.01% |
| 3 | 98% | 1.9% | 0.1% |
| 4 | 95% | 4.9% | 0.1% |
| 5 | 90% | 9.9% | 0.1% |
| 6 | 98% | 1.5% | 0.5% |
| 7 | 95% | 4.5% | 0.5% |
| 8 | 90% | 9.5% | 0.5% |
| 9 | 97% | 2% | 1% |
| 10 | 95% | 4% | 1% |
| 11 | 90% | 9% | 1% |
| 12 | 95% | 3% | 2% |
| 13 | 90% | 8% | 2% |
| 14 | 85% | 13% | 2% |

### Prednisone and Prednisolone

Prednisone may be used to treat ocular inflammation. Ophthalmic prednisolone reduces the irritation, redness, burning, and swelling of eye inflammation caused by chemicals, (uveitis) heat, radiation, infection, allergy, or foreign bodies in the eye. It sometimes is used after eye surgery. Dosage schedule is exemplarily 3-5 drops daily, or 1-3 drops three times daily, until symptoms dissipate.

TABLE 2
Prednisone or Prednisolone

| | | | Prednisone or |
|---|---|---|---|
| Example No. | Squalane | Mineral Oil | prednisolone |
| 1 | 98% | 1.99% | 0.01% |
| 2 | 90% | 9.99% | 0.01% |
| 3 | 98% | 1.9% | 0.1% |
| 4 | 95% | 4.9% | 0.1% |
| 5 | 90% | 9.9% | 0.1% |
| 6 | 98% | 1.5% | 0.5% |
| 7 | 95% | 4.5% | 0.5% |
| 8 | 90% | 9.5% | 0.5% |
| 9 | 97% | 2% | 1% |
| 10 | 95% | 4% | 1% |
| 11 | 90% | 9% | 1% |
| 12 | 95% | 3% | 2% |
| 13 | 90% | 8% | 2% |
| 14 | 85% | 13% | 2% |

### Antibiotics

Erythromycin is an antibiotic commonly prescribed for treating conjunctivitis. Ciprofloxacin is an antibiotic commonly prescribed for treating keratitis caused by two of the most clinically significant forms of ocular bacteria, S. aureus and P. aeruginosa. Dosage schedule is exemplarily 3-5 drops daily, evenly spaced depending on severity until symptoms dissipate or altered by a physician.

TABLE 3
Erythromycin or ciprofloxacin

| | | | Erythromycin; |
|---|---|---|---|
| Example No. | Squalane | Mineral Oil | ciprofloxacin |
| 1 | 98% | 1.99% | 0.01% |
| 2 | 90% | 9.99% | 0.01% |
| 3 | 98% | 1.9% | 0.1% |
| 4 | 95% | 4.9% | 0.1% |
| 5 | 90% | 9.9% | 0.1% |
| 6 | 98% | 1.5% | 0.5% |
| 7 | 95% | 4.5% | 0.5% |
| 8 | 90% | 9.5% | 0.5% |
| 9 | 97% | 2% | 1% |
| 10 | 95% | 4% | 1% |
| 11 | 90% | 9% | 1% |
| 12 | 95% | 3% | 2% |
| 13 | 90% | 8% | 2% |
| 14 | 85% | 13% | 2% |

In other compositions, squalane is the only or the main ingredient of a delivery vehicle for ocular therapeutic/treatment substances, particularly including substances for treatment of the skin around the eyes. Contemplated compositions, disclosed below, are all effectively devoid of water, preservatives, emulsifiers and dispersing agents. The compositions each consist essentially of squalane in combination with a pharmaceutical or therapeutic substance, many for skin treatment, in an amount by weight of 0.01% to 5% of the weight of the entire composition. The compositions all include squalane in an amount of at least 80%, and preferably at least 95%, volume/volume. As discussed above, the squalane inhibits dry eye, which effect may be useful to at least some patients to which the composition is applied. Mineral oil or a mixture of mineral oils may be included in an amount of 1% to about 19% volume/volume, but preferably 0% to 5% volume/volume. Where the payload pharmaceutical or therapeutic substance is hydrophobic, the composition may include mineral oil or a mixture of mineral oils in an amount no greater than that necessary to solubilize the hydrophobic substance. In many preferred embodiments, however, the composition consists only of squalane (95% volume/volume and more) with small amounts of the pharmaceutical or treatment substance. See first example for each of the payload therapeutic substances listed below.

Any pharmaceutical substance or therapeutic agent used for treating ocular or peri-ocular tissue in a mammal may be delivered by squalane either alone or in combination with a small amount of mineral oil(s), for instance 1% to 10% and preferably up to 5%. The pharmaceutical substance or therapeutic agent is included in an amount that is conventional in treatment compositions or somewhat less owing to the extended delivery benefits of squalane discussed below.

At least some of the exemplary individual therapeutic agents or substances below constitute family members of more generic therapeutic agents or substances identified hereinabove. The family memberships are known to those versed in the pharmaceutical and chemical therapeutic arts.

### Tacrolimus

A composition for the treatment of atopic dermatitis and other adnexal tissue in pets and humans consists of squalane with Tacrolimus in an amount of 0.03% to 0.05% by weight.

Another composition for the same treatment consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) Tacrolimus in an amount of 0.03% to 0.05% by weight.

### Loteprednol

A composition for the treatment of ocular inflammation and eye lid and facial skin disorders consists of squalane with Loteprednol in an amount of 0.3 to 0.5% by weight, in suspension.

Another composition for the same treatment consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) Loteprednol in an amount of 0.3 to 0.5% by weight.

### Hydrocortisone

A composition for the treatment of ocular and adnexal and facial allergic and inflammatory skin conditions consists of squalane with hydrocortisone in an amount of 1% to 2% by weight.

Another composition for the same treatment consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) hydrocortisone in an amount of 1% to 2% by weight.

### Dexamethasone

A composition for the treatment of ocular and adnexal and facial allergic and inflammatory skin conditions consists of squalane with Dexamethasone in an amount of 0.5% to 1.0% by weight.

Another composition for the same treatment consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) Dexamethasone in an amount of 0.5% to 1.0% by weight.

### Titanium Dioxide

An eyelid composition for absorbing or blocking UVA rays consists of squalane with titanium dioxide powder of nanoscale particle size, in an amount as extant in conventional titanium dioxide sunscreens.

Another composition for the same purpose and application consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) titanium dioxide powder of nanoscale particle size, in an amount as extant in conventional titanium dioxide sunscreens.

### Zinc Oxide

An eyelid composition for absorbing or blocking UVB rays consists of squalane with zinc oxide powder of nanoscale particle size, in an amount as extant in conventional zinc oxide preparations for UVB blockage.

Another composition for the same purpose and application consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) zinc oxide powder of nanoscale particle size, in an amount as extant in conventional zinc oxide preparations for UVB blockage.

### Tobramycin

A composition for the treatment of ocular and peri-ocular skin infections consists of squalane with the antibiotic Tobramycin in an amount of about 3% by weight (e.g., 2.5% to 3.5%).

### Bacitracin

A composition for the treatment of ocular and peri-ocular tissue infections consists of squalane with the antibiotic Bacitracin in an amount of about 3% by weight (e.g., 2.5% to 3.5%).

Another composition for the same treatment consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) Bacitracin in an amount of about 3% by weight (e.g., 2.5% to 3.5%).

### Cefalexin

A composition for treating ocular and peri-ocular tissue for antibacterial purposes consists of squalane with Cefalexin in an amount of about 3% by weight (e.g., 2.5% to 3.5%).

Another composition for the same purpose and application consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) Cefaloxin in an amount of about 3% by weight (e.g., 2.5% to 3.5%).

### Retin-A

An eyelid composition mainly for treating acne and wrinkles consists of squalane with Retin-A or tretinoin in an amount as extant in retin-A and retinol preparations for such applications.

Another composition for the same purpose and application consists of (i) squalane in an amount of at least 80% volume/volume and preferably at least 95% volume/volume, (ii) mineral oil or a mixture of mineral oils in an amount of up to about 20%, and preferably in an amount of 0% to 5%, volume/volume, and (iii) the active ingredient of Retin-A or tretinoin, in an amount as extant in conventional preparations.

### Prostaglandins

As indicated above, squalane (possibly with small amounts of mineral oil, say 1% to 5%) may be used as a delivery vehicle of prostaglandins. The family of prostaglandins includes Latonaprost, Travaprost, bimatoprost, latanprostene (Vyzulta). Any of these may be delivered in a squalane composition in a conventional amount between 0.1% and 5% by weight of the entire composition, for glaucoma therapy. As indicated below, the dosages or weight percentages of the prostaglandins or other therapeutic agents or payload substances can even be reduced relative to conventional dosages or weight percentages owing to the longer viability of squalane in maintaining migration of the therapeutic agents or payload substances into the patient, particularly through the skin.

Where a composition as disclosed herein is applied to peri-ocular skin, the squalane of the delivery vehicle will have the effect of at least reducing or inhibiting dry eye even though the payload ingredient, as indicated above, is intended for skin treatment only. This effect is of special benefit should the target condition occur in combination with dry eye.

The compositions above are applied by devices as described elsewhere herein, namely, applicators such as rollers, pads, and brushes in the case of skin application and eye droppers in the case of application to the eye proper.

Because squalane is not irritating to the eye, using squalane as a delivery vehicle or main portion of the delivery vehicle has the benefit that medication can be administered either once, preferably at bedtime, or twice daily instead of 4 times as when the vehicle is water. Water evaporates and the therapeutic substance or payload dries on the skin, in the case of dermal application. In contrast, squalane maintains its consistency and fluidic consistency for a much longer period, thereby enabling a greater portion of the chemical or therapeutic payload substance to absorb into the patient. With squalane as the principal delivery vehicle (optionally with small amounts of mineral oil), the dosages or weight percentages of the chemical therapeutic agents may be reduced in addition to a reduction in the frequency of application. The same benefit applies to suspensions in squalane for treating facial conditions as well.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof

What is claimed is:

1. A method for treating an ophthalmic or per-ocular skin condition in a mammal in need thereof, the method consisting of administering to an eye or surrounding tissues of said mammal in need thereof therapeutically effective amounts of a composition consisting essentially of:
squalane in an amount of at least 95% volume/volume of the composition, wherein the squalane inhibits dry eye; and
a chemical treatment substance in an amount by weight of 0.01% to 5% of the weight of the entire composition,
the composition being effectively devoid of water, preservatives, emulsifiers and dispersing agents.

2. The method defined in claim 1 wherein the ophthalmic or per-ocular skin condition is taken from the group consisting of atopic dermatitis and other adnexal tissue condition, ocular inflammation and eye lid and facial skin disorders, ocular and adnexal and facial allergic and inflammatory skin conditions, ocular and adnexal and facial allergic and inflammatory skin conditions, exposure to UVA or UVB radiation (prophylactic protection), ocular and peri-ocular skin infections, and acne and skin wrinkles.

3. The method defined in claim 2 wherein the chemical treatment substance is taken from the group consisting of 0.03% to 0.05% Tacrolimus by weight for pets and humans with atopic dermatitis and other adnexal tissue, 0.3% to 0.5% Loteprednol suspensions by weight for ocular inflammation and eye lid and facial skin disorders, 1% to 2% Hydrocortisone by weight for ocular and adnexal and facial allergic and inflammatory skin conditions, 0.5% to 1.0% Dexamethasone by weight for ocular and adnexal and facial allergic and inflammatory skin conditions, titanium dioxide powder of nanoscale particle size as eye lid sunscreen from UVA rays, zinc oxide as UVB sunscreen, about 3% tobramycin by weight as antibiotic for ocular and peri-ocular skin infections, about 3% bacitracin by weight as antibiotic to eye and peri-ocular tissue, and 3% Cefalexin by weight for ocular and per-ocular antibacterial use, wherein the weight percentage for each member of the group is by weight of the entire composition, and Retin-A in a conventional weight percentage or an amount reduced relative to extant amounts.

4. The method defined in claim 1 wherein the chemical treatment substance is a prostaglandin.

5. The method defined in claim 1 wherein the chemical treatment substance is taken from the group consisting of Retin-A, titantium dioxide powder and zinc oxide.

6. A method for treating ocular or peri-ocular tissue in a mammal in need thereof consisting of administering to said mammal in need thereof effective amounts of a composition consisting of squalane, wherein the squalane inhibits dry eye, the composition including squalane in an amount of at least 95% volume/volume, optionally including a mineral oil or mixture of mineral oils and also including a treatment agent or substance, the composition being effectively devoid of water, preservatives, emulsifiers and dispersing agents.

7. The method defined in claim 6, wherein said treatment agent or substance is taken from the group consisting of Tacrolimus, Loteprednol, hydrocortisone Dexamethasone, titanium dioxide powder, zinc oxide, tobramycin, bacitracin, Cefalexin, and Retin-A.

8. The method defined in claim 7, wherein Tacrolimus if present in said composition is in an amount of 0.03% to 0.05% by weight of the entire composition, Loteprednol if present in said composition is in an amount of 0.3% to 0.5% by weight of the entire composition, hydrocortisone if present in said composition is in an amount of 1% to 2% by weight of the entire composition, Dexamethasone if present in said composition is in an amount of 0.5% to 1.0% by weight of the entire composition, tobramycin if present in said composition is in an amount of 0.5% to 1.0% by weight of the entire composition, bacitracin if present in said composition is in an amount of about 3% by weight of the entire composition, Cefalexin if present in said composition is in an amount of about 3% by weight of the entire composition, and Retin-A if present in said composition is in a conventional weight percentage or an amount reduced relative thereto.

9. The method defined in claim 6 wherein the administering of said composition comprises topically applying an effective amount thereof to an eye or per-ocular tissue of the mammal.

10. The method defined in claim 6 wherein the treatment agent or substance is a prostaglandin.

11. The method defined in claim 1 wherein the treatment agent or substance is taken from the group consisting of Retin-A, titantium dioxide powder and zinc oxide.

12. A method for treating ocular or peri-ocular tissue in a mammal in need thereof consisting of administering to an eye or surrounding tissues of said mammal in need thereof therapeutically effective amounts of a composition consisting essentially of squalane in combination with an amount of a treatment substance taken from the group consisting of 0.03% to 0.05% Tacrolimus by weight for pets and humans with atopic dermatitis and other adnexal tissue, 0.3% to 0.5% Loteprednol suspensions by weight for ocular inflammation and eye lid and facial skin disorders, 1% to 2% Hydrocortisone by weight for ocular and adnexal and facial allergic and inflammatory skin conditions, 0.5% to 1.0% Dexamethasone by weight for ocular and adnexal and facial allergic and inflammatory skin conditions, titanium dioxide powder of nanoscale particle size as eye lid sunscreen from UVA rays, zinc oxide as UVB sunscreen, Retin-A for treatment of acne and skin wrinkles, about 3% tobramycin by weight as antibiotic for ocular and peri-ocular skin infections, about 3% bacitracin by weight as antibiotic to eye and peri-ocular tissue, and 3% Cefalexin by weight for ocular and per-ocular antibacterial use, percentage by weight of the entire composition, the composition including squalane in an amount of at least 80% volume/volume, the composition being effectively devoid of water, preservatives, emulsifiers and dispersing agents.

13. The method defined in claim 12 wherein the composition includes mineral oil or mixture of mineral oils in an amount of 1% to 10% volume/volume.

14. The method defined in claim 12 wherein the administering of said composition includes applying said composition to an eye or eyelid of said mammal.

15. The method defined in claim 12 wherein the composition includes squalane in an amount of at least 95% volume/volume.
